(19) United States
(12) Patent Application Publication
(10) Pub. No.: US 2011/0071398 A1
(43) Pub. Date: Mar. 24, 2011

(54) THREE-DIMENSIONAL PROBE APPARATUS

(76) Inventors: Won Soon Hwang, Hanam-si (KR); Yong Cheol Hyeon, Seoul (KR); Seong Rae Kim, Anyang-si (KR)

(21) Appl. No.: 12/881,062

(22) Filed: Sep. 13, 2010

(30) Foreign Application Priority Data
Sep. 22, 2009 (KR) 10-2009-0089458

(51) Int. Cl. A61B 8/14

(52) U.S. Cl. 600/459

(57) ABSTRACT

An ultrasound probe includes an elongated member having a longitudinal axis and a distal housing; a transducer having a transducing surface, the transducer pivotally disposed in the housing and configured so that the transducing surface is capable of selectively rotating between a first position perpendicular to the longitudinal axis and a second position at least collinear with the longitudinal axis; a transmission unit coupled to the transducer to rotate the transducer between the first and second positions; and a location detector coupled to the transmission unit to provide a signal corresponding to an orientation of the transducer between the first and second positions.

### CROSS REFERENCE TO RELATED APPLICATION

This application claims the benefit of Korean Patent Application No. 10-2009-0089458, filed on Sep. 22, 2009, the entire contents of which are incorporated herein by reference.

### BACKGROUND OF THE INVENTION

1. Field of the Invention

The present invention relates to a three-dimensional probe apparatus and, more particularly, to a three-dimensional probe apparatus for an ultrasonic diagnostic apparatus that generates internal images of a patient body using ultrasound waves.

2. Description of the Related Art

Generally, an ultrasonic diagnostic apparatus refers to a non-invasive apparatus that irradiates an ultrasound signal from a surface of a patient body towards a target internal organ beneath the body surface and obtains an image of a monolayer or blood flow in soft tissue from information in the reflected ultrasound signal (ultrasound echo-signal). The ultrasonic diagnostic apparatus has been widely used for diagnosis of the heart, the abdomen, the urinary organs, and in obstetrics and gynecology due to various merits thereof such as small size, low price, real-time image display, and high stability through elimination of radiation exposure, as compared with other image diagnostic systems, such as X-ray diagnostic systems, computerized tomography scanners (CT scanners), magnetic resonance imagers (MRIs), nuclear medicine diagnostic apparatuses, and the like.

The ultrasonic diagnostic apparatus includes a cart-shaped main body for receiving main components thereof, a probe for transmitting and receiving ultrasound signals, a control panel having various switches and keys for inputting commands for manipulation of the apparatus, and a display unit for displaying an image of an ultrasonic diagnosis result.

The probe includes a transducer that converts electrical signals into sound signals or vice versa. The transducer includes an ultrasound wave vibrator assembly composed of a set of ultrasound wave vibrators, which sends ultrasound signals to a target to obtain an image of the target using the signals reflected from the target.

In recent years, with development of image processing techniques, an ultrasonic diagnostic apparatus has been developed to display a three-dimensional ultrasonic image. In such an ultrasonic diagnostic apparatus, the probe obtains the image of a three-dimensional region using a transducer which transmits and receives to the ultrasound signals while moving along a preset locus.

In such a probe, since the transducer can move only along a restricted locus in an advancing direction of the probe, that is, a probing direction, the probe can obtain only an image in one direction if it does not change the probing direction. Therefore, there is a need to provide a probe apparatus that overcomes such a problem.

It should be noted that the above description is provided for understanding of the background of the invention and is not a description of a conventional technique well-known in the art.

### SUMMARY OF THE INVENTION

The present invention is conceived to solve the problems of the related art as described above, and an aspect of the invention is to provide a three-dimensional probe apparatus configured to obtain images in many directions without changing a probing direction.

In accordance with one aspect of the invention, a three-dimensional probe apparatus includes a drive unit configured to generate power in a forward or rearward direction; a power transmission unit configured to transmit the power generated by the drive unit; a transducer configured to receive the power from the power transmission unit to rotate to a location based on the received power; and a location detector configured to detect the location of the transducer.

The location detector may include a first detection member coupled to the power transmission unit and configured to move to a location based on the location of the transducer.

The location detector may include a second detection member configured to determine the location of the transducer based on the location of the first detection member.

The first detection member may be magnetized and the second detection member may include a sensor capable of sensing magnetism of the first detection member.

The drive unit may include a drive motor configured to generate the power; a to driving pulley coupled to a shaft of the drive motor; a driven pulley separated from the driving pulley; and a belt member configured to transmit the power from the driving pulley to the driven pulley.

The power transmission unit may include a power transmission member configured to receive the power from the drive unit; a wire member connecting the power transmission unit to the transducer; a first guide member configured to guide the wire member in a first direction; and a second guide member disposed in a different direction from the first guide member and configured to guide the wire member in a second direction.

The wire member may include a wire connected at one end to the power transmission member and at an opposite end to the transducer; and a pin rod coupled to the wire.

The second direction may be perpendicular to the first direction.

The three-dimensional probe apparatus may include a base to which the transducer is rotatably coupled.

The three-dimensional probe apparatus may be an endocavity probe.

In accordance with another aspect of the invention, an ultrasound probe includes an elongated member having a longitudinal axis and a distal housing; a transducer having a transducing surface, the transducer pivotally disposed in the housing and configured so that the transducing surface is capable of selectively rotating between a first position perpendicular to the longitudinal axis and a second position at least collinear with the longitudinal axis; a transmission unit coupled to the transducer to rotate the transducer between the first and second positions; and a location detector coupled to the transmission unit to provide a signal corresponding to an orientation of the transducer between the first and second positions.

The ultrasound probe may include a drive unit configured to drive the transmission unit in response to the signal. The drive unit may include a drive motor.

The transmission unit may be configured to stop at any point between the first and second positions.

The location detector may include a Hall effect sensor. The location detector may include a light emitting diode configured to emit light. The location detector may to include an optical sensor configured to sense light emitted from the light emitting diode.

The ultrasound probe may be an endocavity ultrasound probe.

### BRIEF DESCRIPTION OF THE DRAWINGS

The above and other aspects, features and advantages of the invention will become apparent from the following description in conjunction with the accompanying drawings, in which:

FIG. 1 is a perspective view of a three-dimensional probe apparatus in accordance with one embodiment of the present invention;

FIG. 2 is a partially cut perspective view of the probe apparatus in accordance with the embodiment of the present invention;

FIG. 3 is an enlarged view of part A of FIG. 2;

FIG. 4 is an enlarged view of part B of FIG. 2;

FIG. 5 is a cross-sectional view of FIG. 4;

FIG. 6 is a block diagram of the three-dimensional probe apparatus of FIG. 2;

FIG. 7 shows an operating state of part “A” of FIG. 2;

FIG. 8 shows an operating state of part “B” of FIG. 2; and

FIG. 9 is a cross-sectional view of FIG. 8.

### DETAILED DESCRIPTION OF THE EMBODIMENT

Exemplary embodiments of the invention will now be described in detail with reference to the accompanying drawings. It should be noted that the drawings are not to precise scale and may be exaggerated in thickness of lines or size of components for descriptive convenience and clarity only. Furthermore, terms used herein are defined by taking functions of the invention into account and can be changed according to the custom or intention of users or operators. Therefore, definition of the terms should be made according to overall disclosures set forth herein.

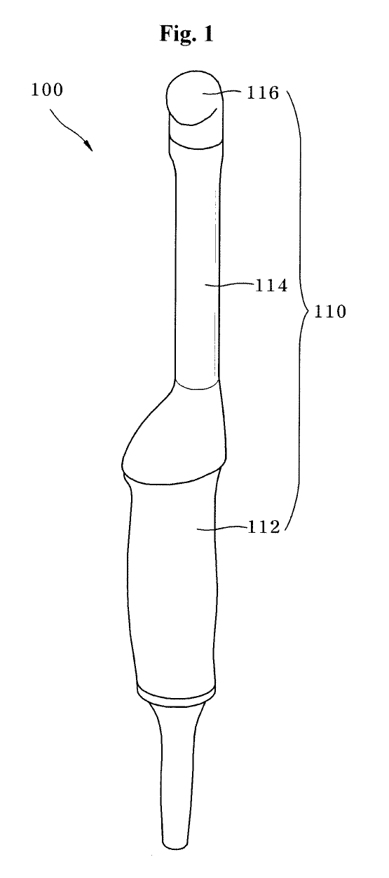

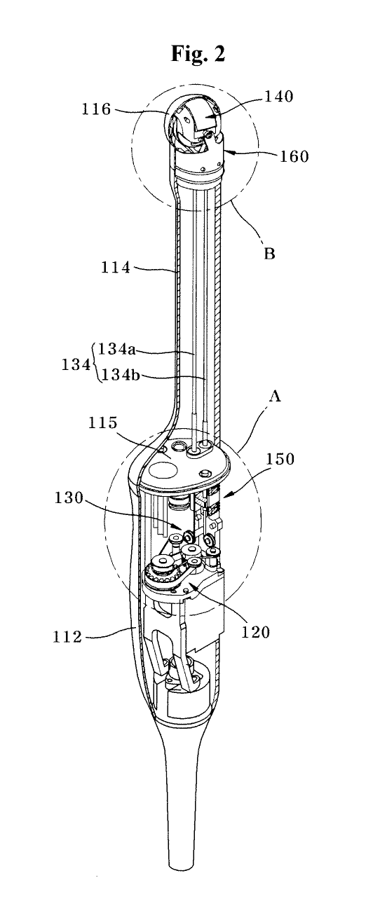

FIG. 1 is a perspective view of a three-dimensional probe apparatus in accordance with one embodiment of the present invention, and FIG. 2 is a partially cut perspective view of the probe apparatus in accordance with the embodiment of the present invention.

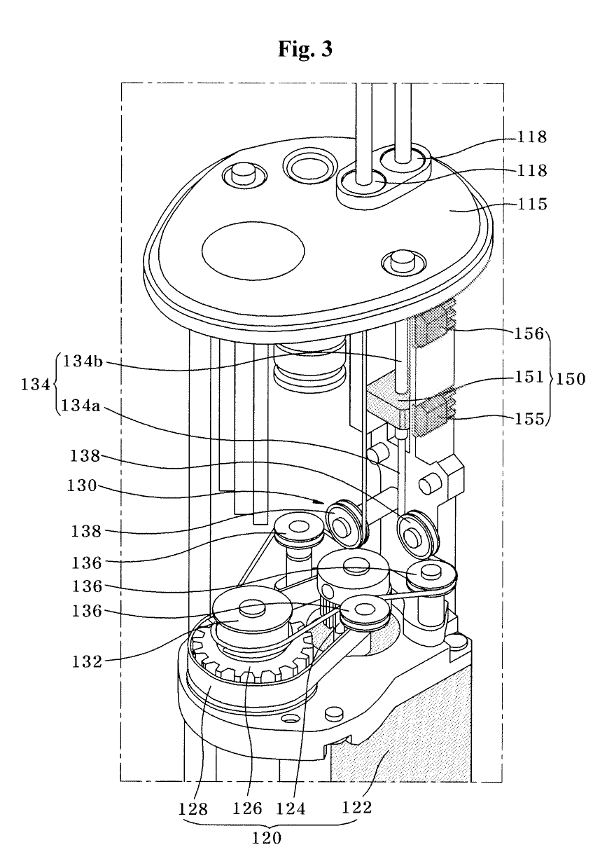

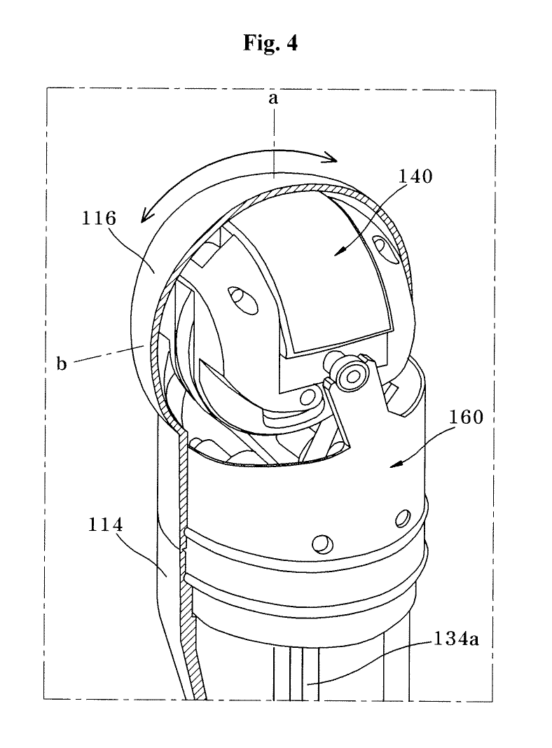

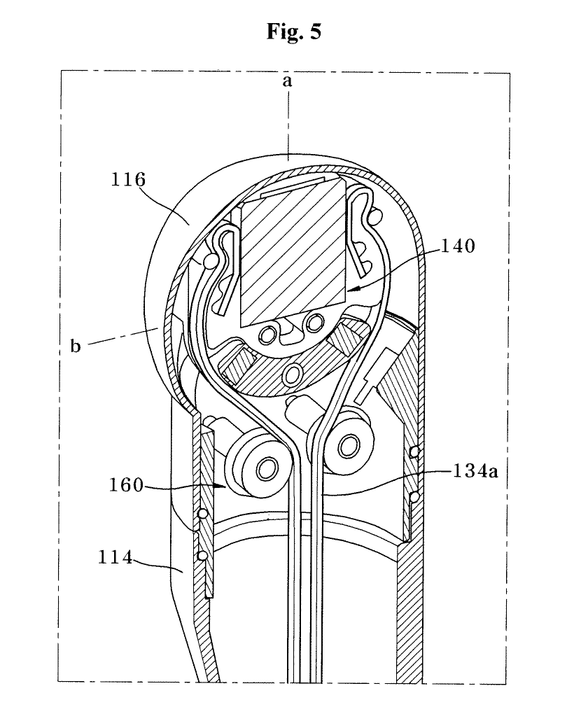

FIG. 3 is an enlarged view of part A of FIG. 2, FIG. 4 is an enlarged view of part B of FIG. 2, and FIG. 5 is a cross-sectional view of FIG. 4.

Referring to FIGS. 1 and 2, a three-dimensional probe apparatus 100 according to one embodiment includes a housing 110, a drive unit 120, a power transmission unit 130, a transducer 140, and a location detector 150.

The housing 110 defines an outer appearance of the probe apparatus 100 according to this embodiment. The housing 110 includes a lower housing 112, an upper housing 114, and a cover 116.

The lower housing 112 defines a lower portion of the housing 110. The lower housing 112 receives the drive unit 120 and the location detector 150.

The upper housing 114 is located on the lower housing 112 and defines an upper portion of the housing 110. The upper housing 114 receives the transducer 140 therein. Oil is also received in the upper housing 114 such that the transducer 140 is immersed in the oil inside the upper housing 114.

A partition 115 is disposed between the upper and lower housings 112 and 114. The partition 115 shields the upper and lower housings 112 and 114 from each other to prevent the oil in the upper housing 114 from leaking into the lower housing 112.

The cover 116 is disposed at an open side of the upper housing 114. The cover 116 is a component to be brought into direct contact with a diagnosis target and covers the upper housing 114 and the transducer 140.

The drive unit 120 generates power in a forward or reverse direction. The drive unit 120 is located inside the lower housing 112 and includes a drive motor 122, a driving pulley 124, a driven pulley 126, and a belt member 128, as shown in FIGS. 2 and 3.

The drive motor 122 generates power. The drive motor 122 can generate a rotational force in the forward or reverse direction.

The driving pulley 124 is coupled to a shaft the drive motor 122. The driving pulley 124 receives the power from the drive motor 122 and is rotated by the rotational force of the drive motor 122 in the forward or reverse direction.

The driven pulley 126 is separated from the driving pulley 124. The driven pulley 126 is rotatably disposed and receives the power from the driving pulley 124 through the belt member 128 which transmits the power from the driving pulley 124 to the driven pulley 126.

The power transmission unit 130 transmits the power in association with the drive unit 120. The power transmission unit 130 is received inside the lower housing 112 and the upper housing 114, and includes a power transmission member 132, a wire member 134, a first guide member 136, and a second guide member 138.

The power transmission member 132 receives the power from the drive unit 120. In this embodiment, the power transmission member 132 is disposed coaxially with the driven pulley 126 and is rotated by rotation of the driven pulley 126 to receive the power from the drive unit 120. The power transmission member 132 can be rotated in the forward or reverse direction by the rotation of the driven pulley 126.

As shown in FIGS. 3 to 5, the wire member 134 connects the power transmission member 132 to the transducer 140. The wire member 134 includes a wire 134a and a pin rod 134b.

The wire 134a connects the power transmission member 132 to the transducer 140. The wire 134a is connected at one side thereof with the power transmission member 132 and at the other side thereof with the transducer 140. In this embodiment, the wire 134a penetrates the partition 115 via a through-hole (reference number omitted) formed in the partition 115. The wire 134a is moved while being wound around or unwound from the power transmission member 132 by forward or rearward rotation of the power transmission member 132 to thereby move the transducer 140.

The pin rod 134b is provided to the wire 134a. The pin rod 134b is located at the middle of the wire 134a and moved together with the wire 134a. The pin rod 134b is disposed to be inserted into the through-hole in the partition 115 and has a sufficient length so as not to be deviated from the through-hole while moving together with the wire 134a.

In this embodiment, the through-hole is provided therein with an oil-seal 118 such that the pin rod 134 is brought into close contact with the oil-seal 118 inside the through-hole. Such close contact between the pin rod 134b and the oil-seal 118 keeps the through-hole in a sealed state to thereby prevent the oil received in the upper housing 114 from leaking through the through-hole.

The first guide member 136 guides the wire member 134 connected to the power transmission member 132 to move in a first direction. The first guide member 136 is separated from the power transmission member 132 and is disposed to be rotated in the same direction as the power transmission member 132. In this embodiment, the first direction is a tangential direction to the rotating direction of the power transmission member 132.

The second guide member 138 is located in a different direction from the first guide member 136 and guides the wire member 134 to move in a second direction. The second guide member 138 is separated from the first guide member 136 and is disposed to be rotated in a direction perpendicular to the rotating direction of the first guide member 136. In this embodiment, the second direction is perpendicular to the first direction.

The wire member 134, which is moved while being wound or unwound by the rotation of the power transmission member 132, may be changed in movement direction toward the second direction by the second guide member 138 to thereby move the transducer 140.

The transducer 140 moves along a preset locus. The transducer 140 is rotatably coupled to a base 160 disposed inside the upper housing 114. While moving along the preset locus, the transducer 140 transmits an ultrasound signal to a target and receives an ultrasound echo signal reflected from the target, thereby enabling realization of a three-dimensional image.

The transducer 140 includes a piezoelectric layer (not shown) in which a piezoelectric material converts electrical signals into sound signals or vice versa while vibrating, a sound matching layer (not shown) reducing a difference in sound impedance between the piezoelectric layer and a target to allow as much of the ultrasound signals generated from the piezoelectric layer as possible to be transferred to the target, a lens layer (not shown) focusing the ultrasound sounds, which travel in front of the piezoelectric layer, onto a predetermined point, and a backing layer (not shown) blocking the ultrasound signals from traveling in the rearward direction of the piezoelectric layer to prevent image distortion.

The transducer 140 receives the power from the power transmission unit 130 to be moved to a plurality of locations for operation. In this embodiment, the transducer 140 may be move to a first location “a” around a second directional axis and a second location “b” around a first directional axis for operation by rotating about a rotational shaft coupled to the base 160.

In this embodiment, the transducer 140 moves right and left along a preset locus at the first location “a” and moves up and down along a preset locus at the second location “b” during operation. In order to allow such movement of the transducer 140, the wire member 134 may have a sufficient length to allow the transducer 140 to be moved not only to the first location “a” but also to the second location “b” during the operation.

The location detector 150 detects a moving location of the transducer 140 to allow the transducer 140 to be moved to the plurality of locations for operation. The location detector 150 includes a first detection member 151, and second detection members 155, 156.

The first detection member 151 is provided to the power transmission unit 130 to detect the moving location of the transducer 140. In this embodiment, the first detection member 151 is provided to the pin rod 134b of the wire member 134 and moves together with the wire member 134.

The location detector 150 includes a plurality of second detection members 155, 156, which are operated in association with the first detection member 151. The plural second detection members 155, 156 are separated from each other by a distance corresponding to the plurality of locations. In this embodiment, the location detector 150 includes two second detection members 155, 156 which are separated from each other by a distance corresponding to the distance between the first location “a” and the second location “b”. The second detection members 155, 156 detect the location of the first detection member 151 to thereby detect the moving location of the transducer 140.

In this embodiment, when the transducer 140 is located at the first location “a”, the first detection member 151 is located to face the second detection member 155, which is located at a lower side of the two second detection members 155, 156. Further, when the transducer 140 is located at the second location “b”, the first detection member 151 is located to face the second detection member 156, which is located at an upper side of the second detection members 155, 156.

For example, the first detection member 151 may be magnetized and the second detection members 155, 156 may be sensors capable of sensing magnetism of the first detection member 151. In this embodiment, the second detection members 155, 156 are hall sensors which detect the magnetism of the first detection member 151 facing one of the second detection members 155, 156, but not limited thereto. Alternatively, a light emitting unit may be provided as the first detection member 151 and optical sensors may be provided as the second detection members 155, 156. As such, the location detector 150 may be realized in various modifications.

The location detector 150 including the first detection member 151 and the second detection members 155, 156 detects the moving location of the transducer 140 by detecting the location of the first detection member 151 through the second detection members 155, 156.

That is, when the second detection member 155 located at the lower side detects the first detection member 151, the location detector 150 detects the moving location of the transducer 140 as the first location “a”, and when the second detection member 156 located at the upper side detects the first detection member 151, the location detector 150 detects the moving location of the transducer 140 as the second location “b”.

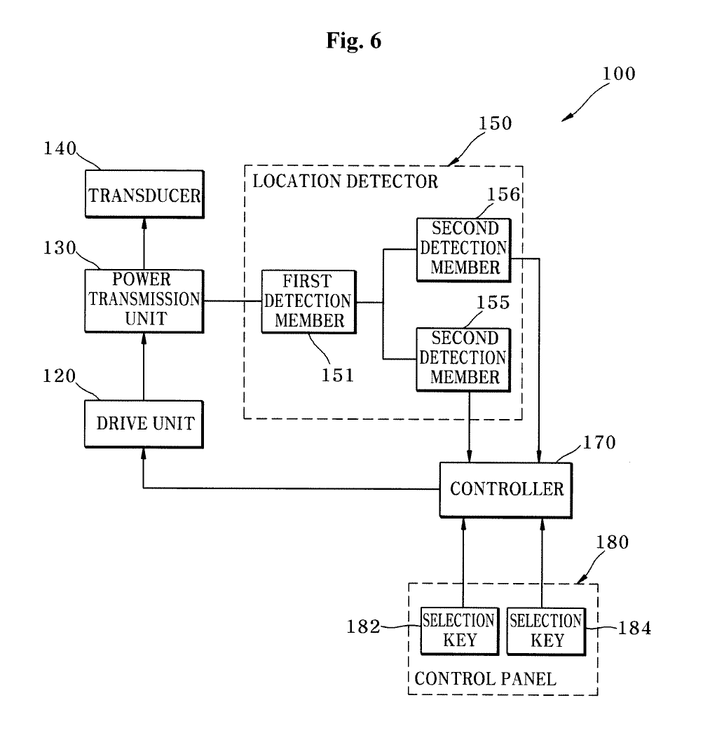

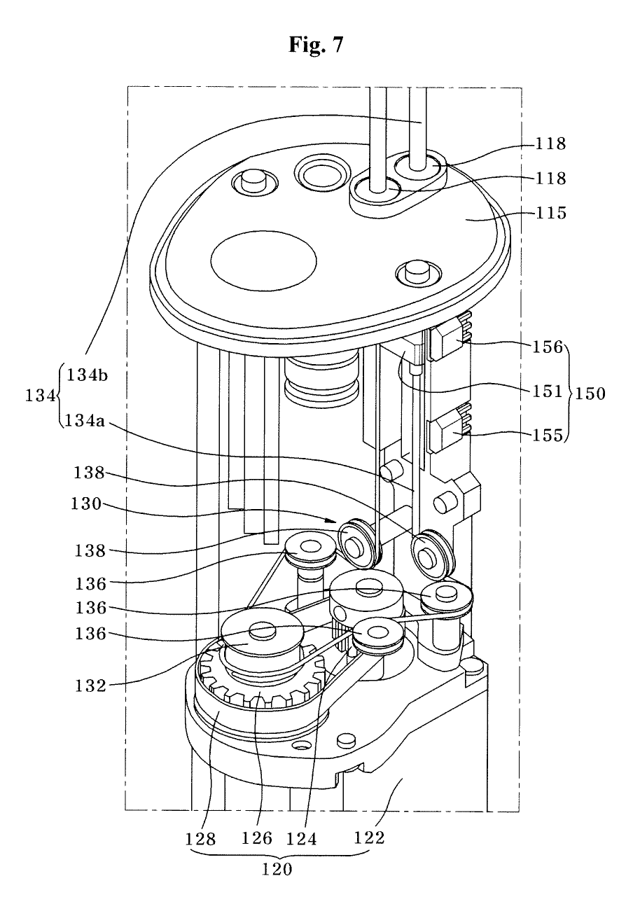

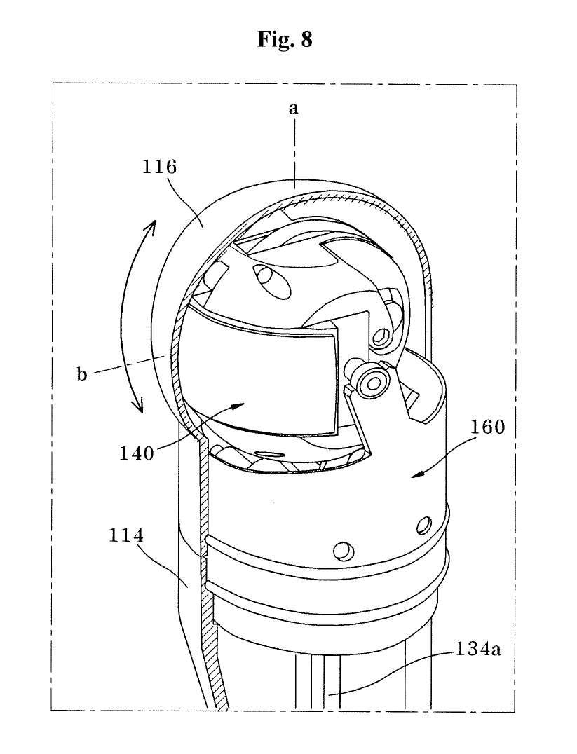

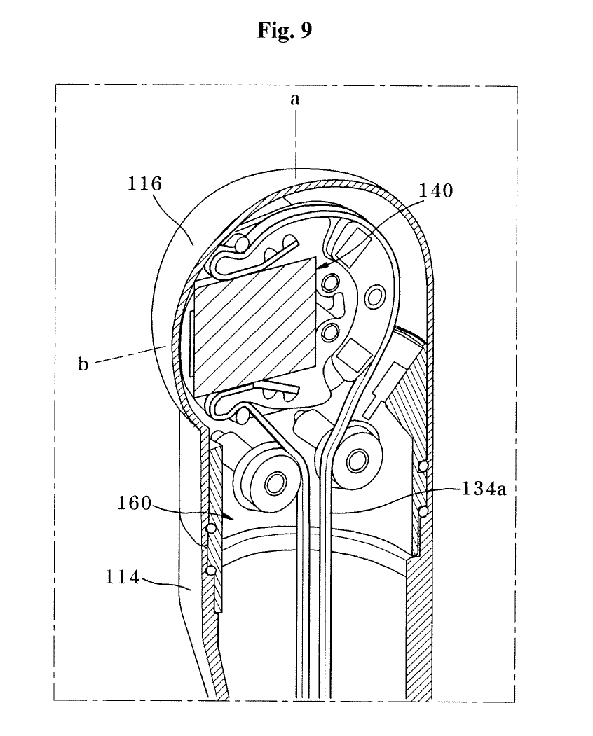

FIG. 6 is a block diagram of the three-dimensional probe apparatus of FIG. 2, FIG. 7 shows an operating state of part “A” of FIG. 2, FIG. 8 shows an operating state of part “B” of FIG. 2, and FIG. 9 is a cross-sectional view of part “B” of FIG. 8.

Next, operation and effect of the three-dimensional probe apparatus 100 according to this embodiment will be described with reference to FIGS. 3 to 9.

First, referring to FIGS. 3 to 6, in the probe apparatus 100 according to this embodiment, the transducer 140 performs a probing operation with respect to a target while moving right and left along a preset locus at a first location “a”, so that a three-dimensional image of the target is obtained. Such movement of the transducer 140 may be achieved by the drive unit 120 and the power transmission unit 130.

Specifically, power generated in the forward or reverse direction by the drive motor 122 of the drive unit 120 is transmitted to the power transmission member 132 of the power transmission unit 130 by connection between the driving pulley 124 and the driven pulley 126 via the belt member 128.

In this embodiment, a forward power is defined as a power in a direction of moving the transducer 140 from the first location “a” to the second location “b”, and a reverse power is defined as a power in a direction of moving the transducer 140 from the second location “b” to the first location “a”.

When receiving the forward or reverse power from the drive unit 120, the power transmission unit 132 transmits the forward or reverse power to the wire member 134. Then, the wire member 134 is moved in the first direction by the first guide member 136 and is moved in the second direction by the second guide member 138 while being wound around or unwound from the power transmission member 132.

While being moved in such a manner, the wire member 134 transmits the forward or reverse power of the drive unit 120 to the transducer 140 to allow the transducer 140 to move right and left along a preset locus at the first location “a”.

The location of the transducer 140 is detected by the location detector 150. Namely, the first detection member 151 is moved to or near a location facing the second detection member 155, which is located at the lower side of the second detection members 155, 156, so that the second detection member 155 detects the location of the first detection member 151 to thereby detect that the transducer 140 is located at or near the first location “a”.

Location information obtained by the location detector 150 is used to define the location of the transducer 140. That is, the location information of the transducer 140 obtained by the location detector 150 is transmitted to a controller 170 in a main body (not shown) of an ultrasonic diagnostic apparatus, and the controller 170 defines the location of the transducer 140 based on the location information of the transducer 140.

When the information is transmitted to the controller 170 from the location detector 150 to inform the controller 170 that the transducer 140 is located at or near the first location “a”, the controller 170 controls the transducer 140 to perform the probing operation in the second direction with reference to the first position “a”.

Then, the transducer 140 performs the probing operation with respect to the target while moving right and left along a preset locus with reference to the first location “a”, and the controller 170 generates a three-dimensional image of the target based on an image of the target obtained by the probing operation in the second direction.

On the other hand, as shown in FIGS. 6 to 9, when the transducer 140 is moved from the first location “a” to a second location “b”, the transducer 140 performs the probing operation with respect to the target while moving up and down along a preset locus with reference to the first location “b”, so that the three-dimensional image of the target is obtained.

Such movement of the transducer 140 to the second location “b” may be performed by user manipulation. For example, a control panel 180 of the ultrasonic diagnostic apparatus may be provided with selection keys 182, 184 for selecting whether the transducer 140 is to be moved to the first location “a” or the second location “b”. When the location of the transducer 140 is selected through manipulation of the selection keys 182, 184 by a user, the controller 170 controls operation of the drive unit 120 according to the user manipulation of the selection keys 182, 184 to allow the transducer 140 to move to the first location “a” or the second location “b”. The location of the transducer 140 at the second location “b” is detected by the location detector 150. In other words, the first detection member 151 is moved to or near a location facing the second detection member 156, which is located at the upper side of the second detection members 155, 156, so that the second detection member 156 detects the location of the first detection member 151 to thereby detect that the transducer 140 is located at or near the second location “b”.

Location information of the transducer 140 obtained by the location detector 150 is transmitted to the controller 170, which in turn defines the location of the transducer 140 based on the location information of the transducer 140.

When the information is transmitted to the controller 170 by the location detector 150 to inform the controller 170 that the transducer 140 is located at or near the second location “b”, the controller 170 controls the transducer 140 to perform the probing operation in the second direction with reference to the second position “b”.

In this embodiment, the location detector 150 includes two second detection members 155, 156 to detect movement of the transducer 140 towards two locations, but it should be noted that this invention is not limited thereto. Alternatively, the location detector 150 may include three or more second detection members to detect movement of the transducer 140 towards two or more locations. As such, it should be noted that the invention can be realized via various modifications.

In this embodiment, the three-dimensional probe apparatus 100 is an endocavity probe. Thus, when the three-dimensional probe apparatus 100 performs a probing operation after being inserted into the body cavity of a person, the probe apparatus can perform, at one location, not only a probing operation with respect to the second direction, that is, an insertion direction, by operation of the transducer 140 around the first location “a”, but also a probing operation with respect to the first direction, that is, a lateral direction, by operation of the transducer 140 around the second location “b”.

According to the embodiment, the transducer 140 may move towards a plurality of locations for operation and the location detector 150 may detect a moving location of the transducer 140 to define the location of the transducer 140 so as to differently control the operation of the transducer 140 in accordance with the moving location of the transducer 140, so that the probe apparatus can perform the probing operation in a plurality of directions at one location without changing the probing direction.

Further, according to the embodiment, the wire member 134 may be guided to move in a plurality of directions including first and second directions, so that a space occupied by the drive unit 120 and the power transmission unit 130 is reduced, thereby reducing the overall size of the probe apparatus.

Although some embodiments have been provided to illustrate the invention in conjunction with the drawings, it will be apparent to those skilled in the art that the embodiments are given by way of illustration only, and that various modifications and equivalent embodiments can be made without departing from the spirit and scope of the invention. Further, the description of the three-dimensional probe apparatus as provided herein is only one example of the invention, and the invention can be applied not only to the three-dimensional probe apparatus but also to a two-dimensional probe apparatus. The scope of the invention should be limited only by the accompanying claims.

What is claimed is:

1. A three-dimensional probe apparatus comprising:
a drive unit configured to generate power in a forward or rearward direction;
a power transmission unit configured to transmit the power generated by the drive unit;
a transducer configured to receive the power from the power transmission unit to rotate to a location based on the received power; and
a location detector configured to detect the location of the transducer.

2. The three-dimensional probe apparatus according to claim 1, wherein the location detector comprises a first detection member coupled to the power transmission unit and configured to move to a location based on the location of the transducer.

3. The three-dimensional probe apparatus according to claim 2, wherein the location detector further comprises a second detection member configured to determine the location of the transducer based on the location of the first detection member.

4. The three-dimensional probe apparatus according to claim 3, wherein the first detection member is magnetized and the second detection member comprises a sensor capable of sensing magnetism of the first detection member.

5. The three-dimensional probe apparatus according to claim 1, wherein the drive unit comprises:
a drive motor configured to generate the power;
a driving pulley coupled to a shaft of the drive motor;
a driven pulley separated from the driving pulley; and
a belt member configured to transmit the power from the driving pulley to the driven pulley.

6. The three-dimensional probe apparatus according to claim 1, wherein the power transmission unit comprises:
a power transmission member configured to receive the power from the drive unit;
a wire member connecting the power transmission unit to the transducer;
a first guide member configured to guide the wire member in a first direction; and
a second guide member disposed in a different direction from the first guide member and configured to guide the wire member in a second direction.

7. The three-dimensional probe apparatus according to claim 6, wherein the wire member comprises:
a wire connected at one end to the power transmission member and at an opposite end to the transducer; and
a pin rod coupled to the wire.

8. The three-dimensional probe apparatus according to claim 6, wherein the second direction is perpendicular to the first direction.

9. The three-dimensional probe apparatus according to claim 1, further comprising:
a base to which the transducer is rotatably coupled.

10. The three-dimensional probe apparatus according to claim 1, wherein the three-dimensional probe apparatus is an endocavity probe.

11. An ultrasound probe comprising:
an elongated member having a longitudinal axis and a distal housing;
a transducer having a transducing surface, the transducer pivotally disposed in the housing and configured so that the transducing surface is capable of selectively rotating between a first position perpendicular to the longitudinal axis and a second position at least collinear with the longitudinal axis;
a transmission unit coupled to the transducer to rotate the transducer between the first and second positions; and
a location detector coupled to the transmission unit to provide a signal corresponding to an orientation of the transducer between the first and second positions.

12. The ultrasound probe of claim 11, further comprising a drive unit configured to drive the transmission unit in response to the signal.

13. The ultrasound probe of claim 12, wherein the drive unit comprises a drive motor.

14. The ultrasound probe of claim 11, wherein the transmission unit is configured to stop at any point between the first and second positions.

15. The ultrasound probe of claim 11, wherein the location detector comprises a Hall effect sensor.

16. The ultrasound probe of claim 11, wherein the location detector comprises a light emitting diode configured to emit light.

17. The ultrasound probe of claim 16, wherein the location detector comprises an optical sensor configured to sense light emitted from the light emitting diode.

18. The ultrasound probe of claim 11, wherein the ultrasound probe comprises an endocavity ultrasound probe.
